(19) United States
(12) Patent Application Publication
(10) Pub. No.: US 2018/0187117 A1
(43) Pub. Date: Jul. 5, 2018

(54) LOW VISCOSITY LUBRICATING POLYOLEFINS

(71) Applicant: TOTAL MARKETING SERVICES, PUTEAUX (FR)

(72) Inventors: Marion COURTIADE, TALUYERS (FR); Julien SANSON, LYON (FR); Alexandre WELLE, COURT-ST-ETIENNE (BE); Martine SLAWINSKI, NIVELLES (BE); Jeroen WASSENAAR, HUIZINGEN (BE)

(73) Assignee: TOTAL MARKETING SERVICES

(21) Appl. No.: 15/740,735

(22) PCT Filed: Jun. 29, 2016
(86) PCT No.: PCT/EP2016/065077

§ 371 Date: Dec. 28, 2017

(30) Foreign Application Priority Data
Jun. 29, 2015 (FR) 1556039

(51) Int. Cl. C10M 105/04; C07C 2/08; C07C 5/05

(52) U.S. Cl. CPC C10M 105/04; C07C 2/08; C07C 5/05; C10M 2203/022; C07C 2531/22; C10N 2240/10; C07C 2523/44; C07C 2531/14; C10N 2230/02

(57) ABSTRACT

A low viscosity oil has more than 50% by weight of 9-methyl-11,13-dioctyltricosane. A lubricating composition having this low viscosity oil as base oil and, optionally, another base oil or an additive. The low viscosity oil has a kinematic viscosity at 100° C., measured by the ASTM D445 standard, of 4 to 8 mm²·s⁻¹. The low viscosity oil can be prepared using a metallocene catalyst, and the low viscosity oil as high performance lubricant can be used for lubrication in the field of motors, hydraulic fluids and gears, in particular bridges and transmissions.

### PRIORITY AND CROSS REFERENCE TO RELATED APPLICATIONS

This application is the U.S. National Phase Application under 35 U.S.C. § 371 of International Application No. PCT/EP2016/065077, filed Jun. 29, 2016, designating the U.S. and published as WO 2017/001442 A1 on Jan. 5, 2017, which claims the benefit of French Application No. FR 1556039, filed Jun. 29, 2015. Any and all applications for which a foreign or a domestic priority is claimed is/are identified in the Application Data Sheet filed herewith and is/are hereby incorporated by reference in their entirety under 37 C.F.R. § 1.57.

### FIELD

The present disclosure generally relates to low viscosity lubricating polyolefins.

### SUMMARY

The invention relates to a low viscosity oil comprising more than 50% by weight of 9-methyl-11,13-dioctyltricosane and to a lubricating composition comprising this base oil and optionally another base oil or additive. This oil of the invention has kinematic viscosity at 100° C., measured as per standard ASTM D445, ranging from 4 to 8 mm²·s⁻¹. The invention also relates to said low viscosity oil prepared following a particular method using a metallocene catalyst, and to the use of this oil as high-performance lubricant for lubrication in the fields of engines, hydraulic fluids, gearboxes, in particular drive axles and transmissions.

### DETAILED DESCRIPTION

In the API classification of base oils, polyalphaolefins (PAOs) are referenced as Group IV base oils. Through a good trade-off between viscosity, volatility and cold start properties, these PAOs are increasingly more used in high performance lubricating formulas. In particular, this better trade-off is highly advantageous in comparison with Group III mineral bases.

In general, PAOs are synthesised from different olefin monomers, particularly from C₆ to C₁₄, monomers, via acid catalysis or in the presence of a metallocene catalyst.

In general, to prepare low viscosity PAO products, notably having kinematic viscosity at 100° C. ranging from 2 to 10 mm²·s⁻¹, measured as per standard ASTM D445 (grades 2 to 10), acid catalysts are used.

Methods are known for preparing PAOs via metallocene catalysis that generally lead to products of high viscosity having kinematic viscosity at 100° C., measured as per standard ASTM D445, ranging from to 40 to 150 mm²·s⁻¹ (grades 40 to 150).

In addition, the needs for high performance lubricants are on the increase. In particular due to increasingly severe conditions of use e.g. due to very high temperatures or mechanical stresses.

Longer oil change times and a reduction in the size of lubricating systems also lead to an increased need for high performance lubricants.

Energy efficiency, and in particular an improvement in the Fuel Eco (FE) of lubricants or reduction in engine fuel consumption, in particular by motor vehicle engines, are objectives of ever increasing importance and lead to an increasing use of high performance lubricants.

High performance lubricants must therefore have improved properties, in particular regarding kinematic viscosity, viscosity index, volatility, dynamic viscosity or cold pour point.

Thermal stability and resistance to oxidation are also properties to be improved for high performance lubricants.

Reduced toxicity and good miscibility with other lubricants or other materials are similarly properties to be sought after for high performance lubricants.

Additionally, methods for preparing improved PAOs must be also be developed, in particular to improve the yield or selectivity of these methods. An improvement in catalytic activity must also be targeted.

Methods for preparing PAOs should also allow control over molecular weight, polydispersity index and the distribution of the formed PAOs.

An improvement in characterization techniques of the different products formed at the time of PAO synthesis is also desirable, in particular for qualitative or quantitative analysis of the formed products.

There is therefore a need for high performance lubricants allowing a solution to be found to all or part of the problems with lubricants in the state of the art.

The invention therefore provides an oil having kinematic viscosity at 100° C., measured as per standard ASTM D445, ranging from 4 to 8 mm²·s⁻¹, and comprising more than 50% by weight of a 1-decene tetramer of formula (I):

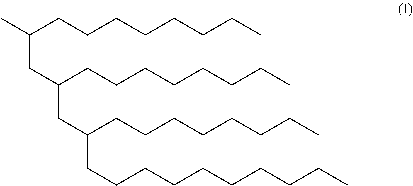 [SMILES: CCCCCCCCCCC(CCCCCCCC)CC(CCCCCCCC)CC(C)CCCCCCCC]

The oil of the invention has particularly advantageous viscosity ranging from 4 to 8 mm²·s⁻¹. More advantageously, the kinematic viscosity of the oil of the invention ranges from 5 to 7 mm²·s⁻¹. Preferably, the kinematic viscosity of the oil of the invention ranges from 5.4 to 6.5 mm²·s⁻¹. More preferably, the kinematic viscosity of the oil of the invention is 5.4 mm²·s⁻¹, 5.5 mm²·s⁻¹, 5.6 mm²·s⁻¹, 5.7 mm²·s⁻¹ or 5.8 mm²·s⁻¹.

Also advantageously, the oil of the invention has a viscosity index higher than 130, or equal to or higher than 140. Preferably the viscosity index of the oil of the invention is between 130 and 180 or between 140 and 160. In general, according to the invention the viscosity index is calculated as per standard ASTM D2270.

Also advantageously, the oil of the invention has volatility measured as per standard CEC L-40-93 that is lower than 6% by weight or lower than 5% by weight. Preferably the volatility of the oil of the invention is between 4 and 6% by weight or between 4.5 and 6% by weight.

Also advantageously, the oil of the invention has dynamic viscosity (CCS) at −35° C., measured as per standard ASTM D5293, that is lower than 4 000 mPa·s. Preferably the dynamic viscosity of the oil of the invention is lower than 3 500 mPa·s or lower than 3 000 mPa·s. According to the invention, the dynamic viscosity of the oil is measured on a rotating dynamic viscometer (cold cranking simulator—CCS).

Also advantageously, the oil of the invention has a mean molecular weight ranging from 300 to 1 000 g/mol, preferably from 400 to 600 g/mol. In general, according to the invention, the mean molecular weight is calculated according to standard ASTM D2502.

Also advantageously, the oil of the invention has a cold pour point of −50° C. or lower, preferably −55° C. or lower or −57° C. lower. In general, according to the invention the cold pour point is measured as per standard EN ISO 3016.

Advantageously, the invention provides an oil combining:
(a) kinematic viscosity at 100° C., measured as per standard ASTM D445, ranging from 5 to 7 mm²·s⁻¹, preferably from 5.4 to 6.5 mm²·s⁻¹, or of 5.4 mm²·s⁻¹, or 5.5 mm²·s⁻¹, or 5.6 mm²·s⁻¹, or 5.7 mm²·s⁻¹ or 5.8 mm²·s⁻¹;
(b) a viscosity index higher than 130, or equal to or higher than 140, or of between 130 and 180, or between 140 and 160;
(c) volatility, measured as per standard CEC L-40-93, lower than 6% by weight or lower than 5% by weight; and
(d) dynamic viscosity (CCS) at −35° C., measured as per standard ASTM D5293, lower than 3 500 mPa·s or lower than 3 000 mPa·s.

Also advantageously, the invention provides an oil combining these properties: (a) and (b); (a) and (c); (a) and (d); (b) and (c); (b) and (d); (c) and (d); (a), (b) and (c); (a), (b) and (d); (a), (c) and (d); (b), (c) et (d).

Preferably, the invention provides an oil combining:
(a) kinematic viscosity at 100° C., measured as per standard ASTM D445, ranging from 5.4 to 6.5 mm²·s⁻¹, or of 5.4 mm²·s⁻¹, or 5.5 mm²·s⁻¹, or 5.6 mm²·s⁻¹, or 5.7 mm²·s⁻¹ or 5.8 mm²·s⁻¹;
(b) a viscosity index of between 130 and 180, or between 140 and 160;
(c) volatility, measured as per standard CEC L-40-93, lower than 5% by weight; and (d) dynamic viscosity (CCS) at −35° C., measured as per standard ASTM D5293, lower than 3 000 mPa·s.

Also preferably, the invention provides an oil combining these properties: (a) and (b); (a) and (c); (a) and (d); (b) and (c); (b) and (d); (c) and (d); (a), (b) and (c); (a), (b) and (d); (a), (c) and (d); (b), (c) and (d).

Advantageously, the oil of the invention comprises 50 to 99% by weight of 1-decene tetramer of formula (I). Also advantageously, the oil of the invention comprises from 60 to 95% by weight or 70 to 90% by weight of 1-decene tetramer of formula (I). Preferably, the oil of the invention comprises at least 65% by weight of 1-decene tetramer of formula (I), or at least 70% by weight of 1-decene tetramer of formula (I). More advantageously, the oil of the invention comprises at least 80% by weight of 1-decene tetramer of formula (I) or at least 90% by weight of 1-decene tetramer of formula (I).

In addition to the 1-decene tetramer of formula (I), the oil of the invention may comprise other oligomers derived from the oligomerisation of 1-decene, in particular saturated oligomers. Preferably, the oil of the invention may comprise at least one other saturated oligomer of 1-decene selected from among:
other saturated tetramers of 1-decene; or
other saturated tetramers of 1-decene, saturated dimers of 1-decene, saturated trimers of 1-decene, saturated pentamers of 1-decene, saturated hexamers of 1-decene; or
the other saturated tetramers of 1-decene, saturated pentamers of 1-decene, saturated hexamers of 1-decene.

Also advantageously, the oil of the invention comprises:
51 to 94.8% by weight of 1-decene tetramer of formula (I);
0.1 to 10% by weight of at least one other saturated tetramer of 1-decene;
0.1 to 10% by weight of at least one saturated trimer of 1-decene;
5 to 25% by weight of at least one saturated pentamer of 1-decene, or of at least one saturated hexamer of 1-decene.

In particular, the oil of the invention comprises:
51 to 94.7% by weight of 1-decene tetramer of formula (I);
0.1 to 10% by weight of at least one other saturated tetramer of 1-decene;
0.1 to 5% by weight of at least one saturated dimer of 1-decene;
0.1 to 10% by weight of at least one saturated trimer of 1-decene;
5 to 25% by weight of at least one saturated pentamer of 1-decene, or of at least one saturated hexamer of 1-decene.

The oil of the invention, as essential characteristic, comprises more than 50% by weight of 9-methyl-11,13-dioctyltricosane, a tetramer of 1-decene of formula (I). Preferably, the oil of the invention comprising more than 50% by weight of 9-methyl-11,13-dioctyltricosane is prepared following a method comprising:
oligomerisation of 1-decene in the presence of hydrogen (H₂), a metallocene catalyst and an activator compound, or in the presence of hydrogen (H₂), a metallocene catalyst, an activator compound and a co-activator compound;
catalytic hydrogenation of the oligomerisation products in the presence of hydrogen (H₂) and a catalyst selected from among a hydrogenation catalyst and a hydrogenation catalyst comprising palladium;
separation, via distillation at reduced pressure, of the fraction of tetramers comprising more than 50% by weight of 1-decene tetramer of formula (I):

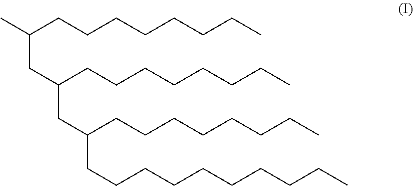 [SMILES: CCCCCCCCCCC(CCCCCCCC)CC(CCCCCCCC)CC(C)CCCCCCCC]

Preferably, oligomerisation of 1-decene is performed in the presence of a metallocene catalyst that is a racemic compound of formula (II)

L(Q¹)(Q²)MR¹R² (II)
where:
   M is a transition metal selected from among titanium, zirconium, hafnium and vanadium, or it is zirconium;
   Q¹ and Q², substituted or unsubstituted are independently a cyclic tetrahydroindenyl group, or Q¹ and Q² are independently a cyclic tetrahydroindenyl group and are linked to form a polycyclic structure;
   L is a divalent C₁-C₂₀-alkyl group bridging Q¹ and Q² or L is a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers thereof, heptylene and isomers thereof, octylene and isomers thereof, nonylene and isomers thereof, decylene and isomers thereof, undecylene and isomers thereof, dodecylene and isomers thereof;
   R¹ and R², substituted or unsubstituted, are independently an atom or a group selected from among hydrogen, halogens (such as Cl and I), alkyl (such as Me, Et, nPr, iPr), alkenyl, alkynyl, haloalkyl, haloalkenyl, haloalkynyl, silylalkyl, silylalkenyls, silylalkynyls, germylalkyl, germylalkenyl, germylalkynyl; or R¹ and R² together with M form a metallocycle having 3 to 20 carbon atoms.
More preferably, the metallocene catalyst is a racemic compound of formula (II) where:
   M is zirconium;
   Q¹ and Q², substituted or unsubstituted are independently a cyclic tetrahydroindenyl group;
   L is a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers, heptylene and isomers thereof, octylene and isomers thereof, nonylene and isomers thereof, decylene and isomers thereof, undecylene and isomers thereof, dodecylene and isomers thereof;
   R¹ and R², substituted or unsubstituted are independently a halogen atom such as Cl and I, or an alkyl group such as Me, Et, nPr, iPr.

Further preferably, the metallocene catalyst is selected from among rac-ethylene bis(tetrahydroindenyl) zirconium dimethyl and rac-ethylene bis(tetrahydroindenyl)zirconium dichloride, in particular rac-ethylene bis(tetrahydroindenyl) zirconium dimethyl.

For the method of the invention, the catalyst is used in activated form for oligomerisation of 1-decene. The invention therefore uses an activator compound when oligomerising 1-decene.

Advantageously, the activator compound is selected from among an alumoxane, an ionic activator and mixtures thereof.

Preferably, for the method of the invention, alumoxane is an oligomeric compound comprising residues of formula-Al(R)—O— where R is independently a cyclic or straight-chain C₁-C₂₀ alkyl group. Preferably, the alumoxane is selected from among methylalumoxane, modified methylalumoxane, ethylalumoxane, isobutylalumoxane and mixtures thereof.

Also preferably, the alumoxane is used in an alumoxane/catalyst molar ratio ranging from 1 to 10 000, preferably ranging from 10 to 3 000 and more preferably from 100 to 1 500. Preferably, for the method of the invention, the activator compound is an ionic activator. The ionic activator can be selected from among dimethylanilinium tetrakis-(perfluorophenyl)borate (DMAB), triphenylcarbonium tetrakis-(perfluorophenyl)borate, dimethylanilinium tetrakis-(perfluorophenyl)aluminate and mixtures thereof. More preferably, the ionic activator is dimethylanilinium tetrakis-(perfluorophenyl)borate (DMAB).

Also preferably, the ionic activator is used in an ionic activator/catalyst molar ratio ranging from 0.5 to 4, and preferably from 0.8 to 1.2.

When oligomerising 1-decene, the method of the invention uses an activator compound. It may also be advantageous to use a co-activator compound, in particular if an ionic activator is used.

Preferably, the co-activator compound is a trialkylaluminium derivative. More preferably, the co-activator compound is selected from among tri-ethyl aluminium (TEAL), tri-iso-butyl aluminium (TIBAL), tri-methyl aluminium (TMA), tri-n-octyl aluminium and methyl-methyl-ethyl aluminium (MMEAL). Advantageously, tri-iso-butyl aluminium (TIBAL) is used in the form of a dispersion possibly ranging from 10 to 60% by weight.

Also preferably, the co-activator compound is used in a co-activator/catalyst molar ratio ranging from 10 to 1 000, preferably from 20 to 200.

Advantageously, the metallocene catalyst and the activator compound, optionally in the presence of a co-activator compound, are placed in contact at a pressure of 1 bar and at a temperature of 20° C.

Advantageously, oligomerisation of 1-decene is conducted for a time ranging from 2 to 300 min. Preferably, oligomerisation time ranges from 5 to 180 min, in particular from 30 to 140 min.

Also advantageously, oligomerisation of 1-decene is conducted in the presence of hydrogen (H₂) at a partial pressure ranging from 0.1 to 20 bar. Preferably the partial pressure of hydrogen ranges from 1 to 6 bar.

Also advantageously, oligomerisation is performed with a hydrogen/1-decene mass ratio higher than 100 ppm or lower than 600 ppm. Preferably, this ratio is between 100 and 600 ppm.

Also advantageously, oligomerisation of 1-decene is conducted at a temperature ranging from 50 to 200° C., preferably from 70 to 160° C. More preferably, the temperature when oligomerising 1-decene ranges from 80 to 150° C., and more preferably from 90 to 140° C. or from 100 to 130° C.

Oligomerisation of 1-denene can be performed in the 1-decene used as medium for the reaction. The reaction is then advantageously conducted in the absence of any solvent. Oligomerisation of 1-decene can also be conducted in a solvent. Preferably, the solvent can be selected from among a straight-chain or branched hydrocarbon, a cyclic or non-cyclic hydrocarbon, an alkylated aromatic compound and mixtures thereof. As preferred solvents for oligomerisation of 1-decene, preference is given to a solvent from among butanes, pentanes, hexanes, heptanes, octanes, cyclopentane, cyclohexane, methylcyclopentane, methylcyclohexane, methylcycloheptane, toluene, xylene and mixtures thereof.

After oligomerisation of 1-decene, the method of the invention applies catalytic hydrogenation of the oligomerisation products. Catalytic hydrogenation of the oligomerisation products is performed in the presence of hydrogen (H₂) and a hydrogenation catalyst.

Preferably, the hydrogenation catalyst is selected from among a palladium derivative, supported palladium derivative, alumina-supported palladium derivative (e.g. on gamma-alumina), nickel derivative, supported nickel derivative, kieselguhr-supported nickel derivative, platinum derivative, supported platinum derivative, cobalt-molybdenum derivative, supported cobalt-molybdenum derivative.

More preferably, the hydrogenation catalyst comprises palladium. One particularly preferred hydrogenation catalyst comprises alumina-supported palladium (e.g. on gamma-alumina).

Also preferably, the pressure of hydrogen (H₂) for catalytic hydrogenation of the oligomerisation products ranges from 5 to 50 bar, more preferably from 10 to 40 bar, in particular from 15 to 25 bar.

After oligomerisation of 1-decene and catalytic hydrogenation of the oligomerisation products, the method of the invention comprises separation via distillation at reduced pressure of the faction of tetramers comprising more than 50% by weight of 1-decene tetramer of formula (I).

Separation via distillation is performed at reduced pressure. Advantageously, separation via distillation is performed in accordance with standard ASTM D5236.

Preferably, for separation via distillation according to standard ASTM D5236, the initial boiling point (IBP) is between 450 and 520° C., preferably between 475 and 495° C. The partial pressure is advantageously lower than 0.67 mbar.

Preferably, separation via distillation according to standard ASTM D5236 allows separation of the fraction of tetramers comprising more than 50% by weight of 1-decene tetramer of formula (I).

Therefore, separation via distillation at reduced pressure allows separation of the fraction of tetramers resulting from oligomerisation of 1-decene, followed by hydrogenation of the oligomerisation products. This fraction of tetramers comprises more than 50% by weight of 1-decene tetramer of formula (I).

In addition to the steps of oligomerising 1-decene, catalytic hydrogenation of the oligomerisation products and separation via distillation at reduced pressure of the fraction of tetramers comprising more than 50% by weight of 1-decene tetramer of formula (I), the method of the invention may advantageously comprise other steps. For example, the method of the invention may also combine all or part of the following steps:
prior preparation of the 1-decene via catalytic oligomerisation of ethylene;
deactivation of the catalyst after oligomerisation of 1-decene or after catalytic hydrogenation of the oligomerisation products;
recycling a fraction of 1-decene dimers (e.g. 9-methyl nonadecane), separated via distillation at reduced pressure, and oligomerisation with 1-decene of this recycled fraction of 1-decene dimers, in the presence of (H₂), a metallocene catalyst and an activator compound, or in the presence of hydrogen (H₂), a metallocene catalyst, an activator compound and a co-activator compound;
a final hydrogenation step of the fraction of tetramers comprising more than 50% by weight of 1-decene tetramer of formula (I) in the presence of hydrogen (H₂) and a catalyst selected from among a hydrogenation catalyst and a hydrogenation catalyst comprising palladium.

The prior preparation of 1-decene via catalytic oligomerisation of ethylene is known per se. It may prove to be particularly advantageous in combination with the other steps of the method of the invention. This prior preparation of 1-decene via catalytic oligomerisation of ethylene notably allows the use of more abundant sources of the starting substrate.

Also, and preferably, once oligomerisation of 1-decene is completed, the method of the invention may comprise deactivation of the catalyst. Deactivation of the oligomerisation catalyst can be carried out after oligomerisation of 1-décène or after catalytic hydrogenation of the oligomerisation products. Preferably, deactivation of the oligomerisation catalyst is performed after oligomerisation of 1-decene and before catalytic hydrogenation of the oligomerisation products.

Advantageously, deactivation of the catalyst is obtained by action of air or water or by means of at least one alcohol or a solution of deactivating agent. Preferably, deactivation of the catalyst is obtained by means of an alcohol e.g. isopropanol.

Also preferably, the method of the invention may comprise a final hydrogenation step of the fraction of tetramers comprising more than 50% by weight of 1-decene tetramer of formula (I). This final hydrogenation is performed in the presence of hydrogen (H₂) and a hydrogenation catalyst.

Preferably, the hydrogen catalyst is selected from among a palladium derivative, supported-palladium derivative, alumina-supported palladium derivative (e.g. on gamma-alumina), nickel derivative, supported nickel derivative, kieselguhr-supported nickel derivative, platinum derivative, supported platinum derivative, cobalt-molybdenum derivative, supported cobalt-molybdenum derivative. More preferably the hydrogenation catalyst comprises palladium. One particularly preferred catalyst comprises alumina-supported palladium (e.g. gamma-alumina). The hydrogenation catalyst is advantageously identical to the hydrogenation catalyst used for the hydrogenation following after oligomerisation of 1-decene.

Advantageously, for final hydrogenation, the pressure of hydrogen (H₂) ranges from 5 to 50 bar or from 10 to 40 bar, preferably from 15 to 25 bar.

Also advantageously, for final hydrogenation, the hydrogenating time is between 2 and 600 min, preferably between 30 and 300 min.

Advantageously, for final hydrogenation, the temperature ranges from 50 to 200° C. or from 60 to 150° C. Preferably the temperature ranges from 70 to 140° C. or from 80 to 120° C.

Preferably, the oil of the invention is prepared according to a method wherein:
oligomerisation of 1-decene is conducted for a time ranging from 2 to 300 min or from 5 to 180 min or from 30 to 140 min; or
oligomerisation of 1-decene is conducted in the presence of hydrogen (H₂) at partial pressure ranging from 0.1 to 20 bar, or from 1 to 6 bar; or
oligomerisation is conducted with a hydrogen/1-decene mass ratio higher than 100 ppm or lower than 600 ppm, or it is between 100 and 600 ppm; or
oligomerisation of 1-decene is conducted at a temperature ranging from 50 to 200° C. or from 70 to 160° C. or from 80 to 150° C. or from 90 to 140° C. or from 100 to 130° C.; or
the metallocene catalyst is a racemic compound of formula (II)

L(Q¹)(Q²)MR¹R² (II)
where:
   M is a transition metal selected from among titanium, zirconium, hafnium and vanadium, or it is zirconium;
   Q¹ and Q², substituted or unsubstituted are independently a cyclic tetrahydroindenyl group or Q¹ and Q² are independently a cyclic tetrahydroindenyl group and are linked to form a polycyclic structure;
   L is a divalent C₁-C₂₀-alkyl group bridging Q¹ and Q², or L is a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers thereof, heptylene and isomers thereof, octylene and isomers thereof, nonylene and isomers thereof, decylene and isomers thereof, undecylene and isomers thereof, dodecylene and isomers thereof;
   R¹ and R², substituted or unsubstituted are independently an atom or a group selected from among hydrogen, halogens (such as Cl and I), alkyl (such as Me, Et, nPr, iPr), alkenyl, alkynyl, haloalkyl, haloalkenyl, haloalkynyl, silylalkyl, silylalkenyls, silylalkynyls, germylalkyl, germylalkenyl, germylalkynyl; or R¹ and R² together with M form a metallocycle having 3 to 20 carbon atoms; or
the metallocene catalyst is selected from among rac-ethylene bis(tetrahydroindenyl) zirconium dimethyl and rac-ethylene bis(tetrahydroindenyl)zirconium dichloride; or
oligomerisation of 1-decene is conducted in a solvent selected from among a straight-chain or branched hydrocarbon, cyclic or non-cyclic hydrocarbon, an alkylated aromatic compound and mixtures thereof, or in a solvent selected from among butanes, pentanes, hexanes, heptanes, octanes, cyclopentane, cyclohexane, methylcyclopentane, methylcyclohexane, methylcycloheptane, toluene, xylene and mixtures thereof; or
the activator compound is selected from among an ionic activator and an oligomeric compound comprising residues of formula-Al(R)—O— where R is independently a cyclic or straight-chain C₁-C₂₀ alkyl group; or the activator compound is selected from among methylalumoxane, modified methylalumoxane, ethylalumoxane, isobutylalumoxane and mixtures thereof; or the activator compound is selected from among dimethylanilinium tetrakis(perfluorophenyl)borate (DMAB), triphenylcarbonium tetrakis(perfluorophenyl)borate, dimethylanilinium tetrakis(perfluorophenyl)aluminate and mixtures thereof; or
the co-activator compound is a trialkylaluminium derivative or a compound selected from among tri-ethyl aluminium (TEAL), tri-iso-butyl aluminium (TIBAL), tri-methyl aluminium (TMA), tri-n-octyl aluminium and methyl-methyl-ethyl aluminium (MMEAL); or
deactivation of the catalyst is performed by action of air or water, or by means of at least one alcohol or a solution of deactivating agent; or
the pressure of hydrogen (H₂) for catalytic hydrogenation of the oligomerisation products ranges from 5 to 50 bar or from 10 to 40 bar or from 15 to 25 bar; or
the hydrogenation catalyst is selected from among a palladium derivative, supported palladium derivative, alumina-supported palladium derivative (e.g. on gamma-alumina), nickel derivative, supported nickel derivative, kieselguhr-supported nickel derivative, platinum derivative, supported platinum derivative, cobalt-molybdenum derivative, supported cobalt-molybdenum derivative; or
the pressure of hydrogen (H₂) at final hydrogenation of the majority fraction by weight of 1-decene tetramer of formula (I) ranges from 5 to 50 bar or from 10 to 40 bar or from 15 to 25 bar; or
the hydrogenating time for the final hydrogenation is between 2 and 600 min or between 30 and 300 min; or
final hydrogenation is conducted at a temperature ranging from 50 to 200° C. or from 60 to 150° C. or from 70 to 140° C. or from 80 to 120° C.; or
the hydrogenation catalyst for final hydrogenation of the fraction of tetramers comprising more than 50% by weight of 1-decene tetramer of formula (I), is selected from among a palladium derivative, supported palladium derivative, alumina-supported palladium derivative (e.g. on gamma-alumina), nickel derivative, supported nickel derivative, kieselguhr-supported nickel derivative, platinum derivative, supported platinum derivative, cobalt-molybdenum derivative, supported cobalt-molybdenum derivative.

More preferably, the oil of the invention is prepared according to a method combining all these characteristics.

Preferably, the oil of the invention is prepared following a method comprising:
oligomerisation of 1-decene in the presence of hydrogen (H₂), a metallocene catalyst and an activator compound, or in the presence of hydrogen (H₂), a metallocene catalyst, an activator compound and a co-activator compound;
catalytic hydrogenation of the oligomerisation products in the presence of hydrogen (H₂) and a catalyst selected from among a hydrogenation catalyst and a hydrogenation catalyst comprising palladium;
separation, via distillation at reduced pressure, of the fraction of tetramers comprising more than 50% by weight of 1-decene tetramer of formula (I).

More preferably, the oil of the invention is prepared with a method combining all these characteristics.

More preferably, the oil of the invention is prepared following a method wherein:
oligomerisation of 1-decene is performed for a time ranging from 2 to 300 min or from 5 to 180 min or from 30 to 140 min;
oligomerisation of 1-decene is performed in the presence of hydrogen (H₂) at partial pressure ranging from 0.1 to 20 bar or from 1 to 6 bar;
oligomerisation of 1-decene is conducted with a hydrogen/1-decene mass ratio higher than 100 ppm or lower than 600 ppm, or between 100 and 600 ppm; or
oligomerisation of 1-decene is conducted at a temperature ranging from 50 to 200° C. or from 70 to 160° C. or from 80 to 150° C. or from 90 to 140° C. or from 100 to 130° C.;
the metallocene catalyst is a racemic compound of formula (II)

L(Q¹)(Q²)MR¹R² (II)
where:
   M is a transition metal selected from among titanium, zirconium, hafnium and vanadium, or it is zirconium;
   Q¹ and Q², substituted or unsubstituted are independently a cyclic tetrahydroindenyl group, or Q¹ and Q² are independently a cyclic tetrahydroindenyl group and are linked to form a polycyclic structure;
   L is a divalent C₁-C₂₀-alkyl group bridging Q¹ and Q², or L is a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers thereof, heptylene and isomers thereof, octylene and isomers thereof, nonylene and isomers thereof, decylene and isomers thereof, undecylene and isomers thereof, dodecylene and isomers thereof;
   R¹ and R², substituted or unsubstituted are independently an atom or a group selected from among hydrogen, halogens (such as Cl and I), alkyl (such as Me, Et, nPr, iPr), alkenyl, alkynyl, haloalkyl, haloalkenyl, haloalkynyl, silylalkyl, silylalkenyls, silylalkynyls, germylalkyl, germylalkenyl, germylalkynyl; or R¹ and R² together with M form a metallocycle having 3 to 20 carbon atoms; or
the metallocene catalyst is selected from among rac-ethylene bis(tetrahydroindenyl) zirconium dimethyl and rac-ethylene bis(tetrahydroindenyl)zirconium dichloride;
oligomerisation of 1-decene is performed in a solvent selected from among a straight-chain or branched hydrocarbon, a cyclic or non-cyclic hydrocarbon, an alkylated aromatic compound and mixtures thereof, or in a solvent selected from among butanes, pentanes, hexanes, heptanes, octanes, cyclopentane, cyclohexane, methylcyclopentane, methylcyclohexane, methylcycloheptane, toluene, xylene and mixtures thereof;
the activator compound is selected from among an ionic activator and an oligomeric compound comprising residues of formula —Al(R)—O— where R is independently a C₁-C₂₀ alkyl group, cyclic or straight-chain; or the activator compound is selected from among methylalumoxane, modified methylalumoxane, ethylalumoxane, isobutylalumoxane and mixtures thereof; or the activator compound is selected from among dimethylanilinium tetrakis(perfluorophenyl)borate, triphenylcarbonium tetrakis(perfluorophenyl)borate, dimethylanilinium tetrakis(perfluorophenyl)aluminate and mixtures thereof;
the co-activator compound is a trialkylaluminium derivative or a compound selected from among tri-ethyl aluminium (TEAL), tri-iso-butyl aluminium (TIBAL), tri-methyl aluminium (TMA), tri-n-octyl aluminium and methyl-methyl-ethyl aluminium (MMEAL);
deactivation of the catalyst is obtained by action of air or water, or by means of at least one alcohol or a solution of deactivating agent; or
the pressure of hydrogen (H₂) for catalytic hydrogenation of the oligomerisation products ranges from 5 to 50 bar or from 10 to 40 bar or from 15 to 25 bar;
the hydrogenation catalyst is selected from among a palladium derivative, supported palladium derivative, alumina-supported palladium derivative (e.g. on gamma-alumina), nickel derivatise, supported nickel derivative, kieselguhr-supported nickel derivative, platinum derivative, supported platinum derivative, cobalt-molybdenum derivative, supported cobalt-molybdenum derivative;
the pressure of hydrogen (H₂) for final hydrogenation of the majority fraction by weight of 1-decene tetramer of formula (I) ranges from 5 to 50 bar or from 10 to 40 bar or from 15 to 25 bar;
the hydrogenating time for final hydrogenation is between 2 and 600 min or between 30 and 300 min;
final hydrogenation is conducted at a temperature ranging from 50 to 200° C. or from 60 to 150° C. or from 70 to 140° C. or from 80 to 120° C.;
the hydrogenation catalyst for final hydrogenation of the fraction of tetramers comprising more than 50% by weight of 1-decene tetramer of formula (I), is selected from among a palladium derivative, supported palladium derivative, alumina-supported palladium derivative (e.g. on gamma-alumina), nickel derivative, supported nickel derivative, kieselguhr-supported nickel derivative, platinum derivative, supported platinum derivative, cobalt-molybdenum derivative, supported cobalt-molybdenum derivative;

In addition to the oligomerisation steps of 1-decene, catalytic hydrogenation of the oligomerisation products and separation via distillation at reduced pressure of the fraction of tetramers comprising more than 50% by weight of 1-decene tetramer of formula (I), the method of the invention may advantageously comprise other steps. For example, the method of the invention may also combine all or part of the following steps:
prior preparation of 1-decene via catalytic oligomerisation of ethylene; or
deactivation of the catalyst after oligomerisation of 1-decene or after catalytic hydrogenation of the oligomerisation products; or
recycling a fraction of 1-decene dimers (e-g-9-methyl nonadecane), separated via distillation at reduced pressure, and oligomerisation with 1-decene of this recycled fraction of 1-decene dimers in the presence of hydrogen (H₂), a metallocene catalyst and an activator compound, or in the presence of hydrogen (H₂), a metallocene catalyst, an activator compound and a co-activator compound; or
a final hydrogenation step of the fraction of tetramers comprising more than 50% by weight of 1-decene tetramer of formula (I) in the presence of hydrogen (H₂) and a catalyst selected from among a hydrogenation catalyst and a hydrogenation catalyst comprising palladium.

The invention also relates to the use of an oil of the invention as base oil or as lubricating base oil. This use therefore concerns an oil of low viscosity comprising more than 50% by weight of 9-methyl-11,13-dioctyltricosane, a tetramer of 1-decene of formula (I).

The invention also relates to the use of an oil of the invention to improve the Fuel Eco (FE) of a lubricant. It also relates to use thereof to reduce the fuel consumption of an engine or to reduce the fuel consumption of a motor vehicle engine. These uses also concern an oil of the invention such as defined by its advantageous, particular or preferred characteristics, and by the preparation method thereof.

The invention also relates to a lubricating composition comprising an oil of the invention. This lubricating composition therefore comprises a low-viscosity oil comprising more than 50% by weight of 9-methyl-11,13-dioctyltricosane, a tetramer de 1-decene of formula (I). Advantageously, the composition of the invention comprises at least 10% by weight or at least 20% by weight of an oil of the invention. Also advantageously, the composition of the invention comprises at least 30, 40, 50 or 60% by weight of an oil of the invention. Also advantageously, the composition of the invention comprise from 10 to 50% by weight, preferably from 10 to 40% by weight or form 15 to 30% by weight of at least one base oil of the invention.

Also advantageously, the composition of the invention comprises an oil of the invention and at least one other base oil. It may also comprise an oil of the invention and at least one additive, or else an oil of the invention, at least one other base oil and at least one additive.

The lubricating composition of the invention may comprise an oil of the invention such as defined by its advantageous, particular or preferred characteristics and by the preparation method thereof.

As other base oil combined with the oil of the invention, the composition of the invention may comprise an oil selected from among a Group III oil, Group IV oil, Group V oil, in particular esters and polyalkylene-glycols (PAG).

The lubricating composition of the invention is particularly advantageous for use as high performance lubricant for lubrication in the fields of engines, hydraulic fluids, gearboxes, in particular drive axles and transmissions.

The invention also relates to the use of a lubricating composition of the invention to improve the Fuel Eco (FE) of a lubricant. It also concerns use thereof to reduce the fuel consumption of an engine or to reduce the fuel consumption of a motor vehicle engine.

The different aspects of the invention are the subject of the following examples given for illustrative purposes.

### EXAMPLES

An autoclave reactor was used equipped with an agitator, a temperature control system and inlets for adding nitrogen, hydrogen and 1-decene.

1-decene (produced by TCI or Acros) was used with purity higher than 94%. It was purified on 3 Å and 13× molecular sieves (Sigma-Aldrich). Before use, the molecular sieves were previously dried at 200° C. for 16 hours.

The products were characterized by ¹H NMR and two-dimensional gas phase chromatography (GC×GC).

For NMR, the PAO samples were diluted in deuterated chloroform, and NMR spectra were obtained at 300 K on Bruker 400 MHz spectrometers: ¹H, ¹³C, HMQC (heteronuclear multiple quantum coherence) and HMBC (heteronuclear multiple bond coherence). Two-dimensional chromatography was used in continuous mode using two apolar and polar columns. The entirety of the effluent leaving the first column was separated in the second dimension. Separation of the compounds was governed by volatility on the first column and by specific interactions (π-π type, dipolar interactions, etc) on the second dimension. As a function of their viscosity, the samples were generally diluted twice in heptane. The chromatographic conditions were optimised for elution of the PAOs prepared according to the invention. The samples were analysed by GC×GC with cryogenic modulation (liquid nitrogen), programming of the first oven from 45° C. (5 min) up to 320° C. (20 min) with a ramp of 3° C./min, programming of the secondary oven from 60° C. (5 min) up to 330° C. (20 min) with a ramp of 3° C./min; the columns used under the following conditions:
1^{st} dimension: HP1, 25 m, ID 0.32 mm, film thickness: 0.17 μm;
2^{nd} dimension: BPX-50, 1.5 m, ID 0.1 mm, film thickness: 0.1 μm;
injector: split 100:1, injected volume: 0.1 μl;
detector: FID, 320° C.;
temperature of hot jet: 320° C.;
cold jet programming from 80 to 5%;
modulation period: 4.8 s.

### Example 1

An 8 L autoclave reactor was used. Before use, the reactor was dried at 130° C. under a stream of nitrogen for one hour, and cooled to 110° C. It was then filled with 3 500 mL of 1-decene under a stream of nitrogen. The temperature of the reactor was held at 110° C. and hydrogen (H₂) added in an m/m ratio of H₂/1-decene of 414 ppm.

The catalyst was rac-ethylene bis(tetrahydroindenyl) zirconium dimethyl activated with dimethylanilinium tetrakis(perfluorophenyl)borate (DMAB) in a B/Zr molar ratio of 1.75. Triisobutyl aluminium (TiBAl) was used as co-activator compound in an Al/Zr molar ratio of 200. It allowed trapping of impurities present in the reactor.

Oligomerisation was initiated at the time the activated catalyst was added in a concentration of 17 μM relative to the oligomerisation solution.

After 120 min, 5 mL of isopropanol were added to deactivate the catalyst.

Hydrogenation of the reaction products was performed using an alumina-supported palladium catalyst (5 g of palladium on gamma-alumina at 5% w/w relative to alumina—produced by Alfa Aesar) and hydrogen (H₂) at 20 bar, at a temperature of 100° C., for full hydrogenation (followed by NMR to control removal of unsaturations).

The oligomerisation products and the fraction of tetramers comprising more than 50% by weight of 9-methyl-11,13-dioctyltricosane were then separated via distillation at reduced pressure (0.67 mbar) as per standard ASTM D5236, using a column with 15 theoretical plates having a maximum temperature of 495° C. This distillation in accordance with standard ASTM D5236 allowed the isolation of products having a boiling point of between 475 and 495° C.

The 9-methyl-11,13-dioctyltricosane content of the oil of the invention obtained was 72.73%.

This oil of the invention comprising more than 50% by weight of 9-methyl-11,13-dioctyltricosane has kinematic viscosity at 100° C., measured as per standard ASTM D445, of 5.823 mm²·s⁻¹. The viscosity index of this oil is 144. Its volatility measured as per standard CEC L-40-93 is 4.6% by weight and its dynamic viscosity (CCS) at −35° C., measured as per standard ASTM D5293, is 2 950 mPa·s. Its mean molecular weight is 479 g/mol, calculated as per standard ASTM D2502.

The characteristics of the oil of the invention allow excellent lubricating, rheological properties to be obtained, in particular under cold conditions, as well as oxidation resistance and Fuel Eco properties.

### Example 2—Comparative

Identical measurements and characterizations were performed on a reference commercial oil. This was a PAO oil (Ineos Durasyn 166) prepared from olefins via acid catalysis. This reference PAO oil has kinematic viscosity at 100° C., measured as per standard ASTM D445, of 5.864 mm²·s⁻¹. Its viscosity index is 137. Its volatility measured as per standard CEC L-40-93 is 6.8% by weight and its dynamic viscosity (CCS) at −35° C., measured as per standard ASTM D5293 is 3 870 mPa·s. Its mean molecular weight is 473 g/mol, calculated as per standard ASTM D2502.

Also, the specifications of this commercial oil are the following: kinematic viscosity at 100° C., measured as per standard ASTM D445, of 5.7 to 6.1 mm²·s⁻¹; volatility, measured as per standard CEC L-40-93, lower than 7% by weight.

The oligomers contained in this oil were characterized by ¹H NMR and by two-dimensional gas phase chromatography (GC×GC). The oligomer distribution of this PAO is 34 weight % of the different C₃₀ oligomers, 42 weight % of the different C₄₀ oligomers and 15 weight % of the different C₅₀ oligomers, the remainder being composed of other oligomers.

The method of the invention therefore allows an oil to be prepared having properties equivalent to or higher than those of commercial PAO oils, in particular the viscosity index, volatility or cold start dynamic viscosity are much better for the oils of the invention.

### Example 3: Preparation of a Lubricating Composition of the Invention (1) and of a Comparative Lubricating Composition (1)

Lubricating compositions were prepared by mixing the oil of Example 1 or a known PAO oil with another base oil of Group III, viscosity index-improving polymers and a mixture of additives (dispersants, detergents including sulfonate, friction modifier, antioxidant, pour point improver, anti-wear agent). The lubricating compositions thus prepared are described in Table 1 (weight %).

TABLE 1

| | Composition (1) | Comparative |
|---|---|---|
| | of the invention | composition (1) |
| Group III base oil (grade 4) | 64.09 | 64.09 |
| Group IV base oil PAO 6 (Ineos | 0 | 20 |
| Durasyn 166) | | |
| oil (1) of the invention | 20 | 0 |
| mixture of additives | 9.51 | 9.51 |
| polymers | 6.4 | 6.4 |

The characteristics of the prepared lubricating compositions were evaluated, and the results obtained are given in Table 2.

TABLE 2

| | Composition (1) | Comparative |
|---|---|---|
| | of the invention | composition (1) |
| kinematic viscosity at 100° C. | 8.156 | 8.227 |
| (NF EN ISO 3104) (mm² · s⁻¹) | | |
| viscosity index (ISO 2909) | 172 | 171 |
| HTHS Ravenfield at 150° C. | 2.62 | 2.61 |
| (CEC L-36-A-90/ASTM D4741) | | |
| (mPa · s) | | |
| Noack volatility (CEC L-40-93) | 9.6 | 10.5 |
| (% m/m) | | |
| dynamic viscosity (CCS) | 4 090 | 4 400 |
| at −35° C. (ASTM D5293) | | |
| (mPa · s) | | |

The lubricating compositions comprising the oil (1) of the invention exhibit improved properties compared with the lubricating compositions comprising a known PAO base oil. The cold start dynamic viscosity is lower. Noack volatility is improved.

### Example 4: Preparation of a Lubricating Composition According to the Invention (2) and of a Comparative Lubricating Composition (2)

Lubricating compositions were prepared by mixing the oil of Example 1 or a known PAO oil with another base oil of Group III, viscosity-index improving polymers, and a mixture of additives (dispersants, friction modifier, detergents including sulfonate, antioxidant, pour point improver, anti-wear agent). The lubricating compositions thus prepared are described in Table 3 (weight %).

TABLE 3

| | Composition (2) | Comparative |
|---|---|---|
| | of the invention | composition (2) |
| Group III base oil (grade 4) | 65.7 | 65.7 |
| Groupe IV base oil PAO 6 (Ineos | 0 | 15 |
| Durasyn 166) | | |
| oil (1) of the invention | 15 | 0 |
| mixture of additives | 15.9 | 15.9 |
| polymers | 3.4 | 3.4 |

The characteristics of the prepared lubricating compositions were evaluated, and the results obtained are given in Table 4.

TABLE 4

| | Composition (2) | Comparative |
|---|---|---|
| | of the invention | composition (2) |
| kinematic viscosity at 100° C. | 7.801 | 7.801 |
| (NF EN ISO 3104) | | |
| (mm² · s⁻¹) | | |
| viscosity index (ISO 2909) | 178 | 176 |
| HTHS Ravenfield at 150° C. | 2.60 | 2.63 |
| (CEC L-36-A-90/ASTM D4741) | | |
| (mPa · s) | | |
| Noack volatility (CEC L-40-93) | 10.6 | 11.0 |
| (% m/m) | | |
| dynamic viscosity (CCS) | 4 995 | 5 250 |
| at −35° C. (ASTM D5293) | | |
| (mPa · s) | | |

The lubricating compositions comprising the oil (1) of the invention exhibit improved properties compared with the lubricating composition comprising a known PAO base oil. The viscosity index is higher. Cold start dynamic viscosity is lower. Noack volatility is improved.

### Example 5: Evaluation of Motor Vehicle Engine Lubricating Properties of a Lubricating Composition of the Invention (1) and of a Comparative Lubricating Composition (1)

An EB2 engine was used (PSA Peugeot Citroen) with engine displacement of 1.2 L (maximum power of 60 kW), driven by an electric motor generator

The lubricating composition (1) of the invention and the comparative lubricating composition (1) were compared with a reference lubricating composition (grade SAE 0W-20).

Each friction measurement was performed for about 12 h and allowed detailed mapping of the friction torque induced by each lubricating composition. The tests were carried out in the following sequence:
rinsing the engine with a rinsing oil with added detergents, followed by rinsing with the reference composition;
measurement of the friction torque at 4 temperatures with the reference composition;
rinsing the engine with a rinsing oil with added detergents, followed by rinsing with the lubricating composition to be evaluated;
measuring the friction torque at the 4 temperatures with the lubricating composition to be evaluated;
rinsing the engine with a rinsing oil with added detergents, followed by rinsing with the reference oil;
measuring the friction torque at 4 temperatures with the reference lubricating composition.

The ranges of variation in engine speed and in temperature levels were selected so as to cover the most representative operating points of the NEDC and WLTC type-approval cycles. The 4 temperature levels chosen were coherent with the cycles under consideration. The following set points were used:
engine outlet water temperature: 35° C./50° C./80° C./95° C.±2° C.,
oil temperature ramp: 35° C./50° C./80° C./115° C.±2° C.,
inlet air temperature: 28° C.±2° C.,
exhaust gas counter-pressure: 100 mbar at 5 000 rpm.

The driving torque and the indicated engine operating torque were measured over the selected speed ranges and temperatures. For each temperature, a warm-up phase of 90 min was heeded. Measurement was initiated when the temperatures of oil and water reached the set temperature+/−0.5° C. For each operating point, 4 measurements were taken averaged on 250 revolutions, the measurement of the friction torque on this point corresponding to the mean of these 4 values. A thermal stabilisation time of 5 minutes was observed after each engine speed or temperature ramp.

The friction gain was evaluated for each lubricating composition as a function of temperature and engine speed, then compared with the friction measured for the reference lubricating composition. Friction gains can be positive or negative in which case they are a friction loss. The results of friction gains obtained between the lubricating composition (1) of the invention and the comparative lubricating composition (1) are given in Table 5.

TABLE 5

| engine outlet water temperature/ | |
|---|---|
| oil temperature ramp | friction gain (%) |
| 35° C./35° C. | 1.86 |
| 50° C./50° C. | 1.04 |
| 80° C./80° C. | 0.58 |
| 95° C./115° C. | 0.81 |

It is ascertained that the lubricating composition (1) of the invention allows a major gain in friction compared with the comparative lubricating composition (1) at the different temperatures of use.

On the basis of these friction gains, and after processing via a transfer function, the friction gains resulting from the use of these lubricating compositions were evaluated on the normalised type-approval NEDC and WLTC cycles. This transfer function was based on a vehicle model developed on the SimulationX application (ITI GmbH).This model takes into account tracing of the driving cycle under consideration (speed and gear shift as a function of time), motor and resistive forces applying to the vehicle, the characteristics of the powertrain (engine characteristics, gear ratio, inertia, etc) and data resulting from tests (friction mapping, water and oil temperature rises over the driving cycle under consideration).

The friction gain results obtained between lubricating composition (1) of the invention and the comparative lubricating composition (1) are given in Table 6.

TABLE 6

| | friction gain |
|---|---|
| NEDC cycle gain (%) | 1.17 |
| WLTC cycle gain (%) | 0.97 |

The lubricating composition of the invention therefore allows a major gain in friction compared with the comparative lubricating composition, and therefore allows the envisaging of a major reduction in CO₂ emission

### Example 6: Evaluation of Motor Vehicle Engine Lubricating Properties of a Lubricating Composition According to the Invention (2) and a Comparative Lubricating Composition (2)

An N20 (BMW) engine was used of 2.0 L engine displacement (maximum power of 180 kW), driven by an electric motor generator.

The lubricating composition (2) of the invention and the comparative lubricating composition (2) were compared with a reference lubricating composition (grade SAE 0W-30).

The evaluation conditions were adapted to the conditions of Example 5. The set points applied were the following:
engine outlet water temperature: 40° C./60° C./90° C./110° C.±2° C.
oil temperature ramp: 40° C./60° C./90° C./110° C.±2° C.
inlet air temperature: 21° C.±2° C.
exhaust gas counter-pressure: 40 mbar at 4 000 rpm

The friction gain was evaluated for each lubricating composition as a function of temperature and engine speed, then compared with the friction measured for the reference lubricating composition. The friction gain results obtained between the lubricating composition (2) of the invention and the comparative lubricating composition (2) are given in Table 7.

TABLE 7

| engine outlet water temperature/ | |
|---|---|
| oil ramp temperature | friction gain (%) |
| 40° C./40° C. | 1.25 |
| 60° C./60° C. | 1.22 |
| 90° C./90° C. | 0.86 |
| 110° C./110° C. | 0.77 |

It is ascertained found that the lubricating composition (2) of the invention allows a major friction gain compared with the comparative lubricating composition (2) at the different temperatures of use. On the basis of these friction gains, a reduction in CO₂ emission can be expected.

1. An oil having kinematic viscosity at 100° C., measured as per standard ASTM D445, ranging from 4 to 8 mm²·s⁻¹, comprising more than 50% by weight of 1-decene tetramer of formula (I):

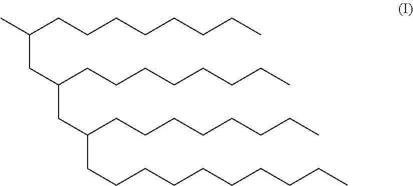 [SMILES: CCCCCCCCCCC(CCCCCCCC)CC(CCCCCCCC)CC(C)CCCCCCCC]

2. The oil according to claim 1, comprising from 50 to 99% by weight of 1-decene tetramer of formula (I).

3. The oil according to claim 1, comprising at least 65% by weight of 1-decene tetramer of formula (I).

4. The oil according to claim 1, further comprising at least one other saturated tetramer of 1-decene.

5. The oil according to claim 1, comprising:
51 to 94.8% by weight of 1-decene tetramer of formula (I);
0.1 to 10% by weight of at least one other saturated tetramer of 1-decene;
0.1 to 10% by weight of at least one saturated trimer of 1-decene;
5 to 25% by weight of at least one saturated pentamer of 1-denene or of at least one saturated hexamer of 1-decene.

6. The oil according to claim 1, wherein:
the kinematic viscosity at 100° C., measured as per standard ASTM D445, ranges from 5 to 7 mm²·s⁻¹; or wherein
the viscosity index is higher than 130.

7. The oil according to claim 1, having volatility, measured as per standard ASTM D6375, lower than 6% by weight.

8. The oil according to claim 1, comprising 1-decene tetramer of formula (I) and prepared by a method comprising:
oligomerizing 1-decene in the presence of hydrogen (H₂), a metallocene catalyst and an activator compound, or in the presence of hydrogen (H₂), a metallocene catalyst, an activator compound and a co-activator compound;
catalytically hydrogenating the oligomerisation products in the presence of hydrogen (H₂) and a catalyst selected from the group consisting of a hydrogenation catalyst and a hydrogenation catalyst comprising palladium;
separating, via distillation at reduced pressure, the fraction of tetramers comprising more than 50% by weight of 1-decene tetramer of formula (I).

9. The oil according to claim 1, comprising 1-decene tetramer of formula (I) and prepared by a method comprising:
oligomerizing 1-decene in the presence of hydrogen (H₂), a metallocene catalyst and an activator compound, or in the presence of hydrogen (H₂), a metallocene catalyst, an activator compound and a co-activator compound;
catalytically hydrogenating the oligomerisation products in the presence of hydrogen (H₂) and a catalyst selected from the group consisting of a hydrogenation catalyst and a hydrogenation catalyst comprising palladium;
separating, via distillation at reduced pressure, the fraction of tetramers comprising more than 50% by weight of 1-decene tetramer of formula (I);
the method further comprising:
prior preparation of 1-decene via catalytic oligomerisation of ethylene; or
deactivation of the catalyst after oligomerisation of 1-decene or after catalytic hydrogenation of the oligomerisation products; or
recycling of a fraction of 1-decene dimers (e.g. 9-methyl nonadecane), separated via distillation at reduced pressure, and oligomerisation with 1-decene of this recycled fraction of 1-decene dimers, in the presence of hydrogen (H₂), a metallocene catalyst and an activator compound, or in the presence of hydrogen (H₂), a metallocene catalyst, an activator compound and a co-activator compound; or
a final hydrogenation step of the fraction of tetramers comprising more than 50% by weight of 1-decene tetramer of formula (I) in the presence of hydrogen (H₂) and a catalyst selected from the group consisting of a hydrogenation catalyst and a hydrogenation catalyst comprising palladium.

10. The oil according to one of claims 1 to 7 claim 1, comprising 1-decene tetramer of formula (I) and prepared by a method comprising:
oligomerizing 1-decene in the presence of hydrogen (H₂), a metallocene catalyst and an activator compound, or in the presence of hydrogen (H₂), a metallocene catalyst, an activator compound and a co-activator compound;
catalytically hydrogenating the oligomerisation products in the presence of hydrogen (H₂) and a catalyst selected from the group consisting of a hydrogenation catalyst and a hydrogenation catalyst comprising palladium;
separating, via distillation at reduced pressure, the fraction of tetramers comprising more than 50% by weight of 1-decene tetramer of formula (I),
wherein:
oligomerisation of 1-decene is performed in the presence of hydrogen (H₂) at partial pressure ranging from 0.1 to 20 bar; or
oligomerisation is performed with a hydrogen/1-decene mass ratio higher than 100 ppm or lower than 600 ppm; the metallocene catalyst is a racemic compound of formula (II)
L(Q¹)(Q²)MR¹R² (II)
wherein:
M is a transition metal selected from the group consisting of titanium, zirconium, hafnium and vanadium;
Q¹ and Q², substituted or unsubstituted are independently a cyclic tetrahydroindenyl group, or Q¹ and Q² are independently a cyclic tetrahydroindenyl group and are linked to form a polycyclic structure;
L is a divalent C₁-C₂₀-alkyl group bridging Q¹ and Q²;
R¹ and R², substituted or unsubstituted are independently an atom or a group selected from the group consisting of hydrogen, halogens, alkyl, alkenyl, alkynyl, haloalkyl, haloalkenyl, haloalkynyl, silylalkyl, silylalkenyls, silylalkynyls, germylalkyl, germylalkenyl, and germylalkynyl; or R¹ and R² together with M form a metallocene having 3 to 20 carbon atoms; or
the activator compound is selected from the group consisting of an ionic activator and an oligomeric compound comprising residues of formula —Al(R)—O— where R is independently a C₁-C₂₀ alkyl group, cyclic or straight-chain; or the activator compound is selected from the group consisting of dimethylanilinium tetrakis(perfluorophenyl)borate (DMAB), triphenylcarbonium tetrakis(perfluorophenyl)borate, dimethylanilinium tetrakis(perfluorophenyl)aluminate and mixtures thereof; or
the co-activator compound is a trialkylaluminium derivative.

11. (canceled)

12. (canceled)

13. A lubricating composition comprising:
at least one base oil defined according to claim 1; or
at least one base oil defined according to claim 1, and at least one other base oil; or
at least one base oil defined according to claim 1, and at least one additive; or
at least one base oil defined according to claim 1, at least one other base oil and at least one additive.

14. The lubricating composition according to claim 13 comprising:
at least 10% by weight or at least 20% by weight or at least 30% by weight or at least 40% by weight or at least 50% by weight or at least 60% by weight of at least one base oil according to claim 1; or
10 to 50% by weight of at least one base oil according to claim 1.

15. (canceled)

16. A method for reducing a fuel consumption of an engine, the method comprising lubricating said engine with an oil according to claim 1.

17. The method according to claim 16, wherein the engine is a motor vehicle engine.

18. The oil according to claim 1, further comprising at least one other 1-decene compound selected from the group consisting of 1-decene, saturated dimers of 1-decene, saturated trimers of 1-decene, saturated pentamers of 1-decene, and hexamers of 1-decene.
